(19) United States
(12) Patent Application Publication
(10) Pub. No.: US 2023/0293612 A1
(43) Pub. Date: Sep. 21, 2023

(54) METHODS AND SYSTEMS FOR THE SELECTIVE EXTRACTION OF PHYTOCHEMICALS

(71) Applicant: Calcanna Extraction Inc., St. John's (CA)

(72) Inventor: Glenn Antle, St. John's (CA)

(21) Appl. No.: 18/018,262

(22) PCT Filed: Jul. 23, 2021
(86) PCT No.: PCT/CA2021/051028

§ 371 Date: Jan. 26, 2023

Related U.S. Application Data
(60) Provisional application No. 63/057,071, filed on Jul. 27, 2020.

(51) Int. Cl. A61K 36/185

(52) U.S. Cl. CPC A61K 36/185; A61K 2236/37; A61K 2236/17; A61K 2236/51

(57) ABSTRACT

The present application relates to the extraction of phytochemicals, more specifically to methods and systems for the selective extraction of phytochemicals from biomass. A method may comprises placing the biomass into a thermal chamber, the thermal chamber being connected with an eductor through a suction connection, and a discharge port of the eductor being connected to a concentrate tank; generating a vacuum in the thermal chamber by circulating a motive fluid through the eductor; heating the biomass to volatilize one or more phytochemicals; condensing the one or more volatilized phytochemicals by contacting the volatized phytochemicals with the motive fluid; and collecting the one or more condensed phytochemicals into the concentrate tank.

### CROSS-REFERENCE TO RELATED APPLICATIONS

The present application claims priority to U.S. patent application No. 63/057,071, which was filed on Jul. 27, 2020, the contents of which are incorporated herein by reference in their entirety.

### FIELD

The present application relates to the extraction of phytochemicals, more specifically to methods and systems for the selective extraction of phytochemicals from biomass.

### BACKGROUND

In the Cannabis industry, extraction of essential Cannabis oils such as THC, CBD, and other cannabinoids, has become a focus for developing smokeless consumable products. Current means of extraction involve solvent or non solvent based methods. Solvent based methods include the use of hydrocarbons, supercritical fluids such as carbon dioxide, and ethanol as solvents. Non-solvent based methods include rosin pressing, dry sifting, or ice water extraction. Each process can produce a different type of recovered product (crude oil, rosin, hash, keif, etc.) depending on the conditions of the set up.

Drying of the biomass is critical for driving up the potency and is usually done at room temperature and at a humidity of between 55-65%. Drying of cannabis results in a reduction of water content from ˜70% by weight when harvested to 10-15% by weight when dried. Large harvests can occupy significant space when drying the biomass. Typical harvest drying times can range from 5 to 15 days. Low THC content materials are problematic for current solvent extraction technologies as it diminishes the yield and thus requires more material to be processed. This ultimately drives up cost and post processing handling. Such drying and processing typically represents around 25-30% of the total cultivation facility footprint. Solvent extraction technologies rely on high potency feedstock to be able to extract as much cannabinoids as possible with as little solvent required. However, solvent extractions add on the resources required, including material, infrastructure and money, as well as on the environmental footprint.

Drying is a critical step for forming a product that can be used for solvent extraction. Too high of a moisture content reduces throughputs and increases the amount of solvent required reducing throughput and increasing cost of operations. Additionally, solvent extraction processes are unable to selectively isolate individual constituents within the essential oils within their extraction step and rather will extract all soluble compounds (i.e. waxes, fats, paraffins). Furthermore, several post processing steps are required to achieve the final product such as winterization. Lastly, in the case of hydrocarbon and ethanol extraction, removal of the solvent from the product is an extremely important step in the process. Hydrocarbons and ethanol are highly flammable substances and are toxic if consumed. As such, residual amounts of solvents in extraction products represent a hazard to consumers.

In summary, there are many drawbacks in the current extraction techniques. For example, solvent extraction may have limited extraction efficiencies and throughputs based on feed stock composition; solvents used can be dangerous (flammable, combustible); some solvent may be trapped in the biomass and unable to be recovered. Non-solvent extraction may present low processing rates, may require large equipment foot print and be labour intensive. As such, there is a need to develop multi-use and versatile technology applicable to drying the harvested crop, to extraction of crude phytochemicals without the need for solvents, selective separation of cannabis components, and requiring limited pre and post-processing.

### SUMMARY

In certain embodiments, the present disclosure applies low temperature heat to a product while maintaining a vacuum to promote volatilization of the phytochemicals to be separated and recovered as individual components from the biomass or as an emulsion in an infused edible oil product.

It has been surprisingly shown herein that the methods and systems of the present application provide for high selectivity of extraction of different phytochemicals which are obtained in high yield and high purity. Comparable methods and systems did not display the same properties, highlighting the surprising results obtained with the methods and systems of the application.

Accordingly, the present application includes a method for extracting one or more phytochemicals from a biomass, the method comprising: placing the biomass into a thermal chamber, the thermal chamber connected to an eductor through a suction connection, and a discharge port of the eductor connected to a concentrate tank; generating a vacuum in the thermal chamber by circulating a motive fluid through the eductor; heating the biomass to volatilize one or more phytochemicals in the biomass; condensing the one or more volatilized phytochemicals by contact of the volatilized phytochemicals with the motive fluid; and collecting the motive fluid and the one or more condensed phytochemicals into the concentrate tank.

The present application also includes a system for the extraction of one or more phytochemicals from a biomass, the system comprising: a thermal chamber for receiving the biomass; an eductor, connected to the thermal chamber through a suction connection, for circulating a motive fluid thereby creating a vacuum within the system, the eductor comprising a discharge port; a heating source for heating the biomass within the thermal chamber to volatilize one or more phytochemicals; a concentrate tank connected to the discharge port of the eductor for recovering the motive fluid and the one or more condensed phytochemicals; wherein the one or more volatilized phytochemicals are condensed by contact with the motive fluid.

In some embodiments, the biomass is selected from the group consisting of flowers, leaves, wood, bark, roots, seeds, peel, and combinations thereof.

In some embodiments, the biomass is selected from the group consisting of cannabis, lavender, peppermint, spearmint, tea tree, patchouli, eucalyptus, and citrus. In some embodiments, the biomass is cannabis.

In some embodiments, the one or more phytochemicals are selected from the group consisting of terpenes, cannabinoids, essential oils or flavonoids.

In some embodiments, the one or more phytochemicals are selected from the group consisting of tetrahydrocannabinolic acid (THCa), tetrahydrocannabinol (THC), cannabidiolic acid (CBDa), cannabidiol (CBD), cannabigerolic acid (CBGa), cannabigerol (CBG), cannabichromenic acid (CBCa), cannabichromene (CBC), cannabinolic acid (CBNa), cannabinol (CBN), cannabigerovarinic acid (CBGVA), tetrahydrocannabivarin carboxylic acid (THCVA), tetrahydrocannabivarin (THCV), cannabidivarinic acid (CBDVA), cannabidivarin (CBDV), cannabichromevarinic acid (CBCVA), cannabichromevarin (CBCV), limonene, linalool, pinene, myrcene, caryophyllene, pulegone, cineole, terpineol, cymene, apigenin, quercetin, annflavin A, sitosterol and combinations thereof.

In some embodiments, the biomass is a raw feedstock, a partially dried feedstock or a dried feedstock.

In some embodiments, the method and system further comprises drying the biomass in the thermal chamber. In some embodiments, drying the biomass comprises heating the biomass at a temperature of 30° C. to 100° C.

In some embodiments, heating the biomass to volatilize one or more phytochemicals is through indirect heating selected from conductive, convective and radiative heat transfer means. In some embodiments, heating the biomass to volatilize one or more phytochemicals is at a temperature from 30° C. to 300° C. In some embodiments, the temperature is near or lower than the boiling point of the one or more phytochemicals.

In some embodiments, after collecting the one or more condensed phytochemicals into the concentrate tank, the one or more condensed phytochemicals are recovered in a recovery tank.

In some embodiments, after recovering the one or more condensed phytochemicals into the recovery tank, heating is carried out at a second temperature to volatilize a second one or more phytochemicals. In some embodiments, the second temperature is near or lower than the boiling point of the second one or more phytochemicals.

In some embodiments, the eductor further comprises a circulation system for circulating the motive fluid into a closed loop. In some embodiments, the circulation system comprises a circulation pump. In some embodiments, the circulation pump collects the motive fluid from the concentrate tank to return the motive fluid to the eductor.

In some embodiments, the vacuum is at 15-29″ HgVac.

In some embodiments, the motive fluid is selected from N₂, CO₂, compressed air, steam, water, oil and ethanol.

In some embodiments, the method and system further comprises separating the motive fluid from the one or more condensed phytochemicals.

The present application further includes use of a method or a system of the present application in the manufacture of an extract.

In some embodiments, the extract is an essential oil from the group consisting of cannabis, lavender, peppermint, spearmint, tea tree, patchouli, eucalyptus, and citrus.

In some embodiments, the extract is a cannabis extract.

The present application also provides an extract obtained by the method or system of the present application

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole

### BRIEF DESCRIPTION OF DRAWINGS

The embodiments of the application will now be described in greater detail with reference to the attached drawings in which:

FIG. 1 is a schematic illustrating the concept of an eductor.

FIG. 2 is a schematic illustrating a system for an infusion process according to some embodiments of the application.

FIG. 3 is a schematic illustrating a system for a direct recovery process with a gas motive fluid according to some embodiments of the application.

FIGS. 4A and 4B is a schematic illustrating a system for a direct recovery process with a polar solvent motive fluid according to some embodiments of the application.

FIG. 5 is a schematic illustrating a system for a direct recovery process with ethanol as motive fluid according to some embodiments of the application.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

The term “and/or” as used herein means that the listed items are present, or used, individually or in combination. In effect, this term means that “at least one of” or “one or more” of the listed items is used or present.

As used in the present application, the singular forms “a”, “an” and “the” include plural references unless the content clearly dictates otherwise.

In embodiments comprising an “additional” or “second” component, such as an additional or second compound, the second component as used herein is chemically different from the other components or first component. A “third” component is different from the other, first, and second components, and further enumerated or “additional” components are similarly different.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The term “suitable” as used herein means that the selection of the particular composition or conditions would depend on the specific steps to be performed, the identity of the components to be transformed and/or the specific use for the elements, but the selection would be well within the skill of a person trained in the art.

The present description refers to a number of chemical terms and abbreviations used by those skilled in the art. Nevertheless, definitions of selected terms are provided for clarity and consistency.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The term “aq.” as used herein refers to aqueous.

The term “phytochemicals” as used herein refers to chemical compounds produced by plants, including for example carotenoids, polyphenols which include phenolic acids, flavonoids, stilbenes/lignans, anthocyanins, flavones, flavanones, isoflavones, catechins, epicatechins, and proanthocyanidins; terpenes/terpenoids, cannabinoids; essential oils; or any other chemicals of plant origin.

The term “biomass” as used herein refers to plant material used as feedstock for further processing.

The term “eductor” as used herein refers to a type of pump which works on the “venturi effect” to pump out air, gas or liquid from a specified area, requiring only a motive fluid (or driving fluid) for its operation, not requiring any moving parts. When the motive fluid is passed through the eductor at the required capacity (which depends on the design of the eductor), a low pressure or vacuum is created, enabling the eductor to suck liquid or gas from a certain area.

The term “volatilization” or “volatilized” used herein refers to the process of the phase transition of a compound between the liquid phase or semi-solid phase to the gaseous phase or vapour. One may also refer to vaporization or evaporation interchangeably.

### II. Methods and Systems of the Application

It has been surprisingly shown herein that the methods and systems of the present application provide for high selectivity of extraction of different phytochemicals which are obtained in high yield and high purity. Comparable methods and systems did not display the same properties, highlighting the surprising results obtained with the methods and systems of the application.

Accordingly, the present application includes a method for extracting one or more phytochemicals from biomass, the method comprising placing the biomass into a thermal chamber, the thermal chamber connected to an eductor through a suction connection, and a discharge port of the eductor connected to a concentrate tank; generating a vacuum in the thermal chamber by circulating a motive fluid through the eductor; heating the biomass to volatilize one or more phytochemicals; condensing the one or more volatilized phytochemicals by contact with the motive fluid; and collecting the motive fluid and the one or more condensed phytochemicals into the concentrate tank.

The present application further includes a system for the extraction of one or more phytochemicals from biomass, the system comprising: a thermal chamber for receiving the biomass; an eductor connected to the thermal chamber through a suction connection, for circulating a motive fluid thereby creating a vacuum within the system, the eductor comprising a discharge port; a heating source for heating the biomass within the thermal chamber to volatilize one or more phytochemicals; a concentrate tank connected to the discharge port of the eductor for recovering the motive fluid and the one or more condensed phytochemicals; wherein the one or more volatilized phytochemicals are condensed by contact with the motive fluid.

In some embodiments, the biomass is selected from the group consisting of flowers, leaves, wood, bark, roots, seeds, peel, and combinations thereof. In some embodiments, the biomass is selected from the group consisting of cannabis, lavender, peppermint, spearmint, tea tree, patchouli, eucalyptus, and citrus. Any biomass comprising desired phytochemicals to be extracted would be suitable and within the purview of a skilled person.

In some embodiments, the one or more phytochemicals are any compounds contained in the biomass, for example the one or more phytochemicals are selected from the group consisting of terpenes, essential oils, cannabinoids and flavonoids. In some embodiments, the one or more phytochemicals are selected from the group consisting of tetrahydrocannabinolic acid (THCa), tetrahydrocannabinol (THC), cannabidiolic acid (CBDa), cannabidiol (CBD), cannabigerolic acid (CBGa), cannabigerol (CBG), cannabichromenic acid (CBCa), cannabichromene (CBC), cannabinolic acid (CBNa), cannabinol (CBN), cannabigerovarinic acid (CBGVA), tetrahydrocannabivarin carboxylic acid (THCVA), tetrahydrocannabivarin (THCV), cannabidivarinic acid (CBDVA), cannabidivarin (CBDV), cannabichromevarinic acid (CBCVA), cannabichromevarin (CBCV), limonene, linalool, pinene, myrcene, caryophyllene, pulegone, cineole, terpineol, cymene, apigenin, quercetin, annflavin A, sitosterol and combinations thereof. In some embodiments, the one or more phytochemicals are selected from the group consisting essential oils from cannabis, lavender, peppermint, spearmint, tea tree, patchouli, eucalyptus, and citrus.

In some embodiments, the biomass is a raw feedstock, a partially dried feedstock or a dried feedstock. The biomass may be dried or partially dried prior to extraction according to the methods and systems of the present application. For example, dried or partially dried biomass may contain about 10-20% of water based on total weight of biomass. In some embodiments, the water content may be 10-15%. However, the biomass may be a raw feedstock, for example containing 60-80% of water based on total weight of biomass. In some embodiments, the water content may be 60-75%, or 65-70%.

In some embodiments, raw feedstock may be directly used and dried directly in the thermal chamber. The direct drying as part of the methods and systems of the present application may reduce overall costs of extraction by reducing necessary infrastructure for drying, reducing time required for drying, reducing required resources such as equipment, energy and money, and thus improving the overall process.

In some embodiments, drying the biomass comprises heating the biomass at a temperature of 30° C. to 100° C. In some embodiments, drying the biomass is made at a temperature between 30 to 90° C., between 30° C. to 70° C., or between 30° C. to 50° C.

In some embodiments, heating the biomass to volatilize one or more phytochemicals is done through indirect heating selected from conductive, convective and radiative heat transfer means. For example, ceramic heaters, heated media such as oil baths, salt baths and sand baths, heated mantles, forced hot air, induction heaters, microwaves, or any heating means know in the art.

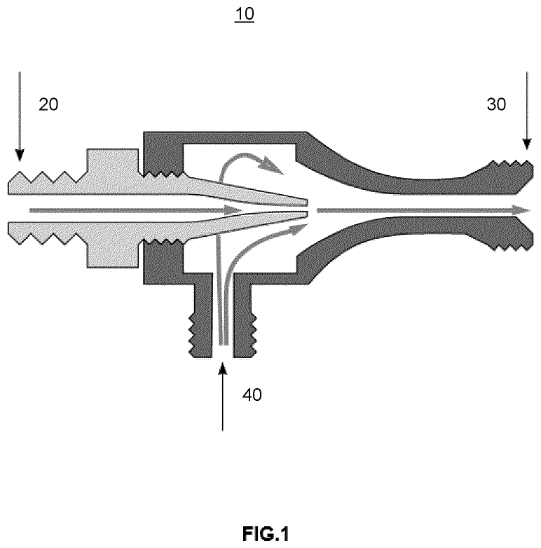

In some embodiments, the thermal chamber is made of glass, ceramic, stainless steel or an alloy of steel, a refractory material or any material that would be food grade compatible. The thermal chamber may be provided with a rotary connection motor to allow the chamber to be rotated for better heat transfer from the indirect heating source. In some embodiments, the thermal chamber is connected with an eductor through a suction connection which is a fixed, non-rotating assembly. An exemplary eductor is shown in FIG. 1. The eductor 10 comprises an input 20 and an ouput 30 (discharge port) for circulating the motive fluid, and thus generating a vacuum within the thermal chamber through the suction connection 40. In some embodiments, the volatilized phytochemicals come into direct contact with a jet stream of motive fluid created by the eductor near the output, where the vapours are condensed, emulsified, and discharged through the discharge port. In some embodiments, the discharge port of the eductor is connected to a concentrate tank for collecting the motive fluid and the one or more condensed phytochemicals.

In some embodiments, the methods and systems may further include additional condensation means or cooling means to accelerate the condensation of the one or more phytochemicals. In some embodiments, the one or more condensed phytochemicals may be further recovered from the concentrate tank into a recovery tank.

In some embodiments, the eductor further comprises a circulation system for circulating the motive fluid into a closed loop. In some embodiments, the circulation system comprises a circulation pump. The closed loop may comprise the eductor, the circulation pump, the concentrate tank and a piping system for continuously circulating the motive fluid from the concentrate tank to the input of the eductor. In some embodiments, the circulation pump collects the motive fluid from the concentrate tank to return the motive fluid to the eductor.

In some embodiments, the vacuum generated by the eductor is at 15-29″ HgVac. In some embodiments, the vacuum generated is between 20 and 28″ HgVac, or between 25 and 28″ HgVac.

In some embodiments, the parameters of the methods and systems may be precisely controlled through various systems including valves, sensors, controllers, etc. For example, thermocouplers may be provided on the system to monitor temperatures at various stages and heating may be adjusted accordingly. In some embodiments, pressure sensors may be provided and the pressure at which the motive fluid enters the eductor and/or the back pressure at which the motive fluid exits the eductor may be controlled, for example through pressure valves, through the speed at which the circulation pump circulates the motive fluid, or any suitable devices known in the art. A skilled person would readily appreciate that various configurations including sensors and controllers may be used. In some embodiments, the monitoring and controlling of the parameters is made automatically according to predetermined extraction threshold for specific biomass and specific phytochemicals.

In some embodiments, the motive fluid is selected from N₂, CO₂, compressed air, steam, water, oil and ethanol, or any suitable motive fluid within the purview of a skilled person in the art. In another embodiment, the oil is coconut oil, olive oil, canola oil, vegetable oil or any other edible oil.

In one embodiment, the motive fluid and the condensed phytochemicals form an emulsion or an infusion. In one embodiment, when the motive fluid is an edible oil (for example, olive oil, vegetable oil, coconut oil, canola oil etc.), the condensed phytochemicals form an emulsion or infusion product with the edible oil.

In some embodiments, heating the biomass to volatilize one or more phytochemicals is conducted at a temperature from 30° C. to 300° C. In some embodiments, the temperature is from 30° C. to 200° C., or from 30° C. to 100° C. In some embodiments, the temperature is selected to be near the boiling point of one or more specific phytochemicals to extract so that the other phytochemicals may remain in the biomass and optionally be later extracted. In some embodiments, the temperature is slightly below the boiling point of the phytochemical, or significantly below the boiling point of the phytochemical.

In some embodiments, after recovering the one or more condensed phytochemicals into the recovery tank, heating is carried out at a second temperature to volatilize a second one or more phytochemicals. For example, selective extraction may be conducted to volatilize THCa or THC, which may be condensed and collected into the recovery tank, and the biomass may further be heated at a second temperature to volatilize CBDa or CBD. For example, the first temperature may be near or below the boiling point of THCa, which is 105° C. In some embodiments, the first temperature may be from 90-105° C., from 80-100° C., from 70-90° C., or from 60-80° C. In some embodiments, the second temperature may be near or below the boiling point of a second phytochemicals. For example, the second temperature may be near or below the boiling point of CBD, which is between 160-180° C. In some embodiments, the second temperature may be from 140-160° C., from 120-140° C., from 100-120° C., or from 80-100° C. In some embodiments, the temperature may be selected to avoid chemical reactions or degradation of phytochemicals, for example the decarboxylation of tetrahydrocannabinolic acid (THCa) into tetrahydrocannabinol (THC) or cannabidiolic acid (CBDa) into cannabidiol (CBD). In some other embodiments, the temperature may be selected to induce such chemical reactions. Thus, the methods and systems of the present application may allow for the selective extraction of different phytochemicals comprised in a biomass by precise control of the temperature and vacuum, as explained above.

For example, parameters for the selective extraction may be based on the boiling points of various phytochemicals, presented in Tables 1 and 2. As such, the temperature and vacuum may be selected and precisely controlled according to parameters of the one or more phytochemical isolate desired for extraction.

TABLE 1
Phytocannabinoids boiling points

| | Concentration | Boiling | |
|---|---|---|---|
| Structure | (% dry weight) | Point | Properties |
| THC | 0.1-25% | 157° C. | Euphoriant |
| 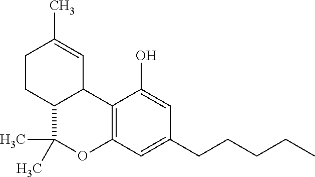 | | | Analgesic Antiinflammatory Antioxidant Antiemetic |
| Λ-9-tetrahydrocannabinol | | | |
| CBD | 0.1-2.89% | 160-180° C. | Anxiolytic |
| 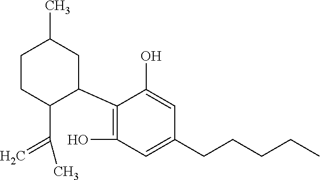 [SMILES: C=C(C)C1CCC(C)CC1c1c(O)cc(CCCCC)cc1O] | | | Analgesic Antipsychotic Antiinflammatory Antioxidant Antispasmodic |
| cannabidiol | | | |
| CBN | 0.0-1.6% | 185° C. | Oxidation breakdown |
| 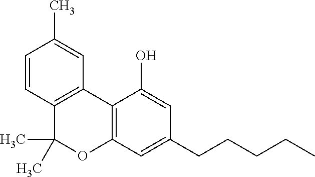 [SMILES: CCCCCc1cc(O)c2c(c1)OC(C)(C)c1ccc(C)cc1-2] | | | product Sedative Antibiotic |
| cannabinol | | | |
| CBC | 0.0-0.65% | 220° C. | Antiinflammatory |
| 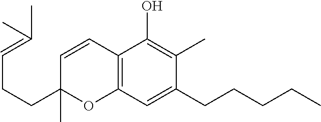 [SMILES: CCCCCc1cc2c(c(O)c1C)C=CC(C)(CCC=C(C)C)O2] | | | Antibiotic Antifungal |
| cannabichromene | | | |
| CBG | 0.03-1.15% | N/A | Antiinflammatory |
| 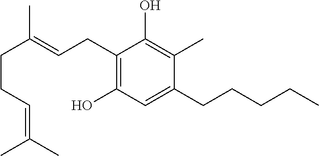 [SMILES: CCCCCc1cc(O)c(C/C=C(\C)CCC=C(C)C)c(O)c1C] | | | Antibiotic Antifungal |
| cannabigerol | | | |
| Δ-8-THC | 0.0-0.1% | 175-178° C. | Resembles Δ-9-THC |
| 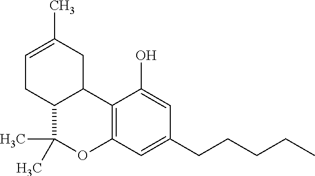 | | | Less psychoactive More stable Antiemetic |
| Λ-8-tetrahydrocannabinol | | | |
| THCV | 0.0-1.36% | <220° C. | Analgesic |
| 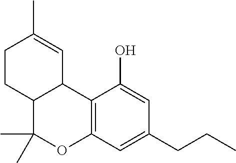 [SMILES: CCCc1cc(O)c2c(c1)OC(C)(C)C1CCC(C)=CC21] | | | Euphoriant |
| tetrahydrocannabivarin | | | |

TABLE 2
Cannabis Terpenoids Boiling Points

| Cannabis Constituent | | Boiling | |
|---|---|---|---|
| Structure | Concentration | Point | Properties |
| d-limonene | 0.14% | 177° C. | Immune potentiator |
| 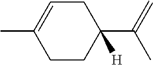 | | | Antidepressant Antimutagenic |
| β-caryophyllene | 0.05% | 119° C. | Antiinflammatory |
| 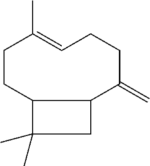 | | | Cytoprotective (gastric mucosa) Antimalarial |
| linalool | 0.002% | 198° C. | Sedative |
| 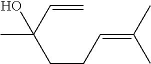 | | | Antidepressant Anxiolytic Immune potentiator |
| β-myrcene | 0.47% | 166-168° C. | Analgesic |
| 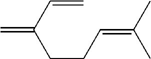 | | | Antiinflammatory Antibiotic Antimutagenic |
| α-pinene | 0.04% | 156° C. | Antiinflammatory |
| 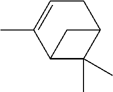 | | | Broncodilator Stimulant Antibiotic Antineoplastic AChE inhibitor |
| 1,8-cineole (eucalyptol) | >0.001% | 176° C. | AChE inhibitor |
| 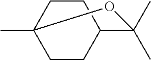 | | | Increases cerebral blood flow Stimulant Antibiotic Antiviral Antiinflammatory |
| | | | Antinociceptive |

In some embodiments, the one or more condensed phytochemicals is recovered from the concentrate tank to a recovery tank while the motive fluid is returned to the eductor. In other embodiments, the motive fluid is recovered together with the condensed phytochemicals. For example, an oil, such as coconut oil, olive oil or an edible oil, may be used as a motive fluid and collected as an infusion. In some embodiments, the method further comprises separating the motive fluid from the one or more condensed phytochemicals to form a desired phytochemical product with high purity and little degradation.

In one embodiment, the process is able to extract at least 80%, or at least 85%, or at least 90% of the desired phytochemical from the biomass. In one embodiment, when the biomass is cannabis, the process is able to extract at least about 85% of THC from the cannabis. In another embodiment, the THC extract is free from organic solvents.

### III. Uses and Extracts of the Application

The methods and systems of the application have been shown useful in selectively extracting phytochemicals from biomass to provide extracts of high purity in a high yield.

Accordingly, the present application includes use of the methods and systems in the manufacture of an extract.

The present application also includes extracts obtained from the present methods and systems.

In some embodiments, the extract is from a cannabis biomass.

### EXAMPLES

The following non-limiting examples are illustrative of the present application.

### General Methods

Infusion process

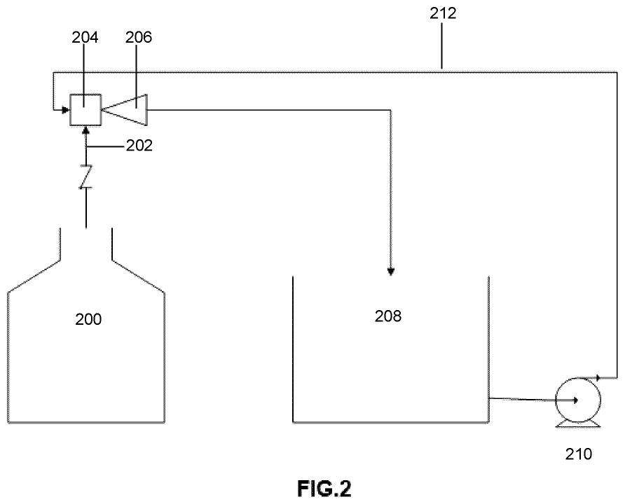

An exemplary system for an infusion process is shown in FIG. 2. A biomass containing desired phytochemicals is introduced into thermal chamber 200 and heated indirectly through means of conductive, convective, or radiative heat transfer means, to a temperature at or slightly lower than the boiling point of the desired phytochemicals while maintaining a temperature less than the combustion temperature of the biomass. The thermal chamber 200 may be provided with a rotary connection motor (not shown) for rotating the thermal chamber whilst allowing the vapour stream to pass through to a fixed non rotating assembly connection (the suction connection 202) to the eductor 204. The eductor 204 is the vacuum source generator as well as the condenser and emulsifier. When the vacuum is generated, the thermal chamber 200 is reduced in pressure, removing oxygen to prevent unwanted oxidative reactions with the phytochemicals being volatilized. As vapours are generated, by the resulting heat transfer from the heating media (not shown) to the thermal chamber 200 thus indirectly to the biomass, they migrate to the suction connection 202 of the eductor, where the vapours come into direct contact with jet stream created by the eductor motive fluid. The vapours are condensed, emulsified, and discharged through the discharge port 206 into a concentrate tank 208. The concentrate tank 208 is connected to a pump 210 which provides the pressure and flow delivery to the eductor 204. As the fluid circulates in the closed loop 212, the concentration of the desired phytochemicals extracted from the biomass increases in the concentrate tank. The concentration of the fluid depends upon the volume of starting fluid, the starting biomass, the concentration of the desired phytochemicals, and the operating temperature of the thermal system. Exemplary conditions parameters are given in Table 3.

TABLE 3

| Item | Operating Range | Units |
|---|---|---|
| Circulating Pump Flowrate | 2-100 | GPM |
| Thermal Chamber Volume | 2-200 | L |
| Rotary Motor | 10-100 | RPM |
| Circulating Pump Pressure | 20-100 | PSIG |
| Circulating Pump Fluid | Oils (vegetable, coconut, | |
| | olive, etc.) & water, | |
| | brine | |
| Thermal Cell Operating | 30-250 | C |
| Temp | | |

This method and system advantageously provide for the direct infusion/emulsification of decarboxylated cannabinoids into an edible oil. No solvents are required; thus, there is no loss of solvent to the biomass during extraction. The precise temperature and pressure control allow for more focused recoveries of cannabinoids and less undesired phytochemicals (for examples plant waxes) which are inevitably recovered during solvent extractions. The number of steps to form the final extract product is considerably reduced as compared to crude oil recovery from solvent extractors.

### Direct Recovery

i. Gas Motive Fluid Liquid Separation

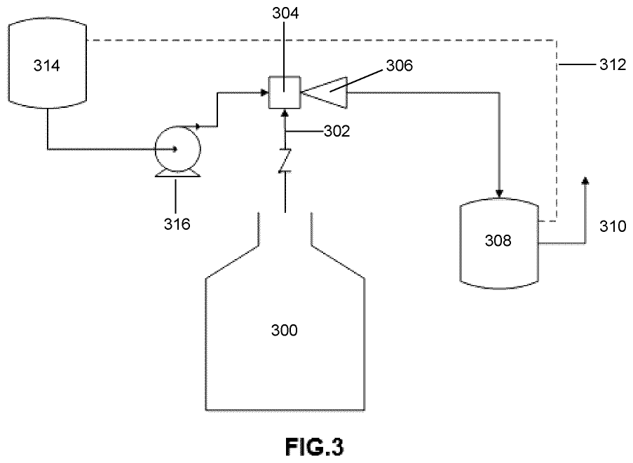

An exemplary system for a direct recovery process is shown in FIG. 3. For crude oil recovery, rather than a liquid motive fluid, a gas or vapour such as N₂, CO₂, compressed air, or steam can be used as the motive fluid. Pressures of the motive fluid may range from 50-150 psig with range of flowrates based on scale of the system and desired vacuum pressure. The thermal chamber 300, the suction connection 302, the eductor 304 and its discharge port 306 are shown. A downstream gas liquid separator 308 is provided as the concentrate tank to allow for cooling of the phytochemicals vapours such that they can sufficiently drop out of the motive fluid and be separated for recovery. A heated recovery tank (not shown) may allow for the condensed oils to be warmed to reduce viscosity. A collection valve (not shown) located on the separator 308 allows for drainage of the product. The gas motive fluid may be discharged to atmosphere through port 310 or be recovered through gas recovery system 312 to gas reservoir 314. A gas pump 316 may then recirculate the motive gas to the eductor 304. Exemplary conditions parameters are given in Table 4.

TABLE 4

| Item | Operating Range | Units |
|---|---|---|
| Gas flowrates | 2-2000 | SCFM |
| Thermal Chamber Volume | 2-200 | L |
| Rotary Motor | 10-100 | RPM |
| Circulating Pump Pressure | 50-150 | PSIG |
| Gas motive fluids | N₂, CO₂, compressed | |
| | air or steam | |
| Thermal Cell Operating | 30-250 | C |
| Temp | | |

This method and system advantageously provide for no solvents being in contact with the biomass at any time, and a single step is necessary to recover crude oil with high efficiencies (<1% residual cannabinoids in the remaining biomass feed stock)

ii. Oil and Motive Fluid Liquid Separation

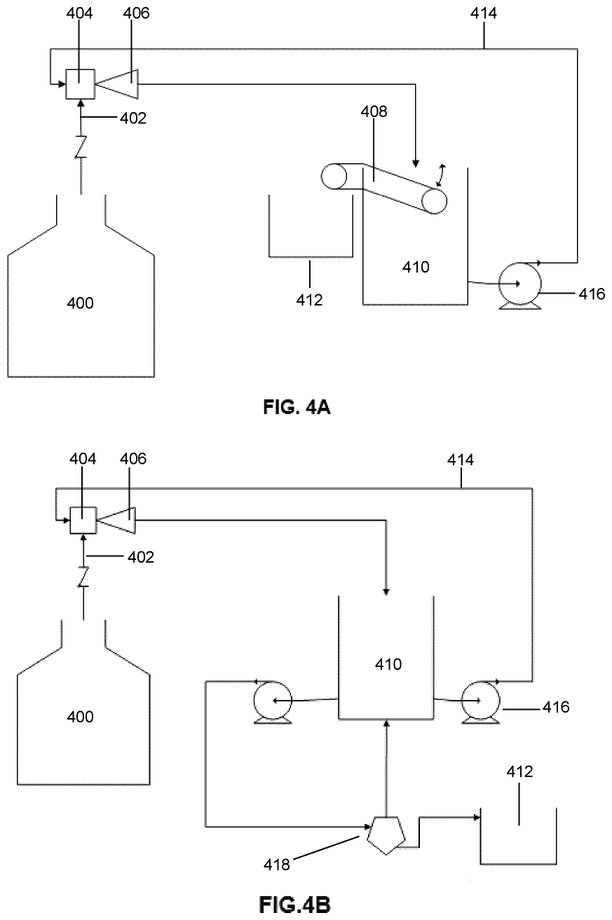

An additional exemplary system is shown in FIG. 4A. This system to separate the crude oil (phytochemicals) uses a polar solvent, such as water, as the motive fluid which may allow the product to separate on the means of density and polarity. The thermal chamber 400, the suction connection 402, the eductor 404 and its discharge port 406 are shown. An oil skimmer 408 at the top of the concentrate tank 410 pulls product containing the phytochemicals onto a non-polar conveyor belt (made of plastic or rubber, for example) which may allow the oily product to stick to the belt as the water would not. The product is recovered and scrapped off the conveyor belt and recovered in a separate recovery tank 412. The operating conditions of the system are the same as above, including a circulation system and circulation pump 416 for circulation of the motive fluid.

A similar exemplary system is shown in FIG. 4B for larger scale applications where an oil skimmer may not be suitable. In such case, a disc stack separator system 418 may be used.

iii. Evaporative Separation

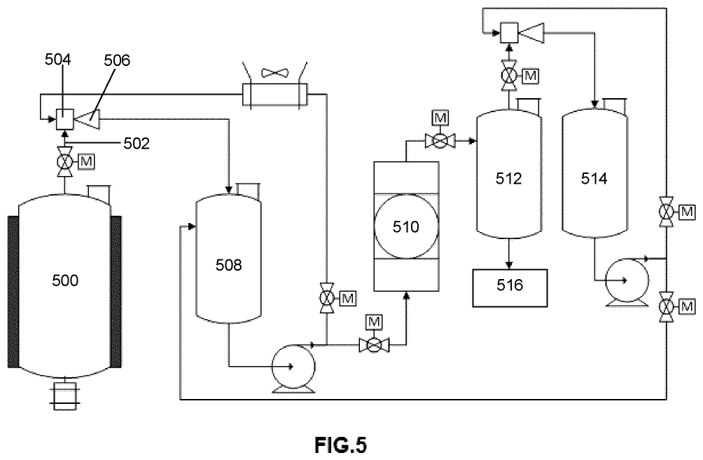

Another exemplary system for recovering phytochemicals from biomass is shown in FIG. 5. The motive fluid used in this process is ethanol. As with all of the other exemplary processes listed above, the thermal chamber 500, the suction connection 502, the eductor 504 and its discharge port 506 are shown and the biomass is heated to promote evaporation of phytochemicals at a reduced system pressure. The volatilized phytochemicals are condensed and concentrated in the ethanol, which are collected in tank 508. Depending upon the amount of solid biomass carried over, a filtration step (filter 510) may be required to remove impurities from the collected mixture. Once the liquor is cleaned using the filter, the ethanol can be removed into ethanol recovery tank 512 and recycled to reservoir 514 and condensed phytochemicals are recovered into a recovery tank 516.

This method and system advantageously provide minimal losses of ethanol during the process. The circulating ethanol can be highly concentrated with crude oil prior to removal of the ethanol thus reducing the amount of ethanol required per mass of feed stock; in theory, all ethanol used can be recovered.

### Example 1

### Results

Cannabis biomass was treated in an exemplary system according to an infusion process of the present application as described in the above Examples. Analyses were conducted for measuring the content of some phytochemicals in the biomass prior the extraction and remaining in the treated biomass. Analysis was performed on HPLC-UV, with a limit of quantitation (LOQ), i.e. the lowest level of analyte that can be accurately quantified, of about 0.10%. Initial content of some cannabinoids and remaining content in the treated biomass are given in Table 5.

TABLE 5

| | Pre-treatment | Post-treatment |
|---|---|---|
| Cannabinoid | content (%) | content (%) |
| Cannabidiol (CBD) | ≤LOQ | ≤LOQ |
| Cannabidiolic acid (CBDa) | ≤LOQ | ≤LOQ |
| Total CBD | ≤LOQ | ≤LOQ |
| Δ 9-tetrahydrocannabinol (Δ 9- | 0.37 | 1.85 |
| THC) | | |
| Δ 9-tetrahydrocannabinolic acid (Δ | 15.78 | 0.4 |
| 9-THCa) | | |
| Total THC | 14.21 | 2.19 |
| Cannabinol (CBN) | ≤LOQ | 5.56 |
| Cannabigerol (CBG) | ≤LOQ | 0.19 |
| Cannabigerolic acid (CBGa) | 0.22 | 0.26 |

These results demonstrate that almost 85% of all THC was extracted from the cannabis biomass, thus showing the high yield of recovery that may be achieved.

### Example 2

### Selectivity

Based on the combination and difference in the boiling points of the cannabinoid isolates THC and CBD at atmospheric pressure and at negative pressure based on −15 inHg vacuum, the selective extraction of the phytochemicals may be achieved. Using a Nomograph, the boiling point (BP) of organic compounds can be estimated when the vacuum pressure is known.

BP_{THC}@760 Torr (1 atm)=about 160° C.

BP_{THC}@381 Torr (−15 inHg)=about 127° C.

BP_{CBD}@760 Torr (1 atm)=about 180° C.

BPCBD@381 Torr (−15 inHg)=about 150° C.

Given the temperature difference in boiling points of the cannabinoid isolates at atmospheric and vacuum pressures, the ability to precisely control processing temperature in the extraction vessel, and the ability to precisely control and vary the amount of vacuum in the extraction system, at −15 inHg or higher, the targeting of the cannabinoid isolate THC can be achieved effectively at about 127° C., 33° C. lower than the BP_{THC} at atmospheric pressure.

Research has also shown that the decarboxylation of the cannabinoids, such as THCa to THC starts to occur at temperature of about 120° C., but can occur over longer periods at temperatures of 80-90° C. (Ref. Tallon et. al. Extraction and fractionation of cannabinoids from Cannabis Sativa). Using the methods described above, water can be removed from the biomass at temperatures of less than 15° C. at 20 Torr, well below the temperatures required for decarboxylation.

### Testing Procedure (Based on CBD Extraction):

The following procedure may be used to determine the selectively of the present methods and systems and recover cannabinoid isolates, specifically THC and CBD.
Accurately recording all operating conditions per the testing data sheets;
Preparing biomass to optimum particle size for extraction (Ref. Tallon et.al.) to between 63 and 125 μm. Use sieve analyses to ensure proper particle size distribution;
Placing 50 g of prepared biomass into the extraction flask;
Setting the desired RPM of the extraction flask to achieve optimum agitation to promote maximum heat transfer;
Setting the temperature of heating bath at to 100° C.
Maintaining temperature for 20 mins, collecting a sample of the motive fluid in 5 min intervals per the sampling protocols;
Increasing the temperature to 105° C. and repeat above steps;
Repeating the procedure by increasing the temperature of the heating bath in intervals of 5° C. until reaching the estimated BP@381 Torr+15° C.
Collecting samples for cannabinoid analyses to include:
   Biomass pretreatment
   Biomass post treatment at each 5° C. interval
   Motive fluid pretreatment;
   Motive fluid post treatment at each 5° C. interval

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

1. A method for extracting one or more phytochemicals from a biomass, the method comprising:
placing the biomass into a thermal chamber, the thermal chamber connected to an eductor through a suction connection, and a discharge port of the eductor connected to a concentrate tank;
generating a vacuum in the thermal chamber by circulating a motive fluid through the eductor;
heating the biomass to volatilize one or more phytochemicals in the biomass;
condensing the one or more volatilized phytochemicals by contact of the volatilized phytochemicals with the motive fluid; and
collecting the motive fluid and the one or more condensed phytochemicals into the concentrate tank.

2. The method of claim 1, wherein the biomass is selected from the group consisting of flowers, leaves, wood, bark, roots, seeds, peel, and combinations thereof.

3. (canceled)

4. The method of claim 1, wherein the biomass is cannabis.

5. The method of claim 1, wherein the one or more phytochemicals are selected from the group consisting of terpenes, cannabinoids, essential oils or flavonoids.

6. The method of claim 4, wherein the one or more phytochemicals are selected from the group consisting of tetrahydrocannabinolic acid (THCa), tetrahydrocannabinol (THC), cannabidiolic acid (CBDa), cannabidiol (CBD), cannabigerolic acid (CBGa), cannabigerol (CBG), cannabichromenic acid (CBCa), cannabichromene (CBC), cannabinolic acid (CBNa), cannabinol (CBN), cannabigerovarinic acid (CBGVA), tetrahydrocannabivarin carboxylic acid (THCVA), tetrahydrocannabivarin (THCV), cannabidivarinic acid (CBDVA), cannabidivarin (CBDV), cannabichromevarinic acid (CBCVA), cannabichromevarin (CBCV), limonene, linalool, pinene, myrcene, caryophyllene, pulegone, cineole, terpineol, cymene, apigenin, quercetin, annflavin A, sitosterol and combinations thereof.

7. (canceled)

8. The method of claim 1, further comprising drying the biomass in the thermal chamber.

9. (canceled)

10. The method of claim 1, wherein heating the biomass to volatilize one or more phytochemicals is through indirect heating selected from conductive, convective and radiative heat transfer means.

11. (canceled)

12. (canceled)

13. (canceled)

14. (canceled)

15. (canceled)

16. The method of claim 1, wherein the eductor further comprises a circulation system for circulating the motive fluid into a closed loop.

17. (canceled)

18. (canceled)

19. The method of claim 1, wherein the vacuum is at 15-29″ HgVac.

20. The method of claim 1, wherein the motive fluid is selected from N₂, CO₂, compressed air, steam, water, oil and ethanol.

21. The method of claim 1, further comprising separating the motive fluid from the one or more condensed phytochemicals.

22. (canceled)

23. (canceled)

24. (canceled)

25. A system for the extraction of one or more phytochemicals from a biomass, the system comprising:
a thermal chamber for receiving the biomass;
an eductor, connected to the thermal chamber through a suction connection, for circulating a motive fluid thereby creating a vacuum within the system, the eductor comprising a discharge port;
a heating source for heating the biomass within the thermal chamber to volatilize one or more phytochemicals;
a concentrate tank connected to the discharge port of the eductor for recovering the motive fluid and the one or more condensed phytochemicals;
wherein the one or more volatilized phytochemicals are condensed by contact with the motive fluid.

26. The system of claim 25, wherein the biomass is selected from the group consisting of flowers, leaves, wood, bark, roots, seeds, peel, and combinations thereof.

27. (canceled)

28. The system of claim 25, wherein the biomass is cannabis.

29. The system of claim 25, wherein the one or more phytochemicals are selected from the group consisting of terpenes, cannabinoids, essential oils or flavonoids.

30. The system of claim 28, wherein the one or more phytochemicals are selected from the group consisting of tetrahydrocannabinolic acid (THCa), tetrahydrocannabinol (THC), cannabidiolic acid (CBDa), cannabidiol (CBD), cannabigerolic acid (CBGa), cannabigerol (CBG), cannabichromenic acid (CBCa), cannabichromene (CBC), cannabinolic acid (CBNa), cannabinol (CBN), cannabigerovarinic acid (CBGVA), tetrahydrocannabivarin carboxylic acid (THCVA), tetrahydrocannabivarin (THCV), cannabidivarinic acid (CBDVA), cannabidivarin (CBDV), cannabichromevarinic acid (CBCVA), cannabichromevarin (CBCV), limonene, linalool, pinene, myrcene, caryophyllene, pulegone, cineole, terpineol, cymene, apigenin, quercetin, annflavin A, sitosterol and combinations thereof.

31. (canceled)

32. The system of claim 25, further comprising drying the biomass in the thermal chamber.

33. (canceled)

34. The system of claim 25, wherein the heating source is an indirect heating source selected from conductive, convective and radiative heat transfer means.

35. (canceled)

36. (canceled)

37. (canceled)

38. The system of claim 25, wherein the eductor further comprises a circulation system for circulating the motive fluid into a closed loop.

39. (canceled)

40. (canceled)

41. The system of claim 25, wherein the vacuum is at 15-29″ HgVac.

42. The system of claim 25, wherein the motive fluid is selected from N₂, CO₂, compressed air, steam, water, oil and ethanol.

43. The system of claim 25, further comprising a separation device for separating the motive fluid from the one or more condensed phytochemicals.

44. (canceled)

45. (canceled)

46. (canceled)

47. (canceled)

48. (canceled)
